(19) United States
(12) Patent Application Publication
(10) Pub. No.: US 2007/0066835 A1
(43) Pub. Date: Mar. 22, 2007

(54) Process for the synthesis of amorphous atorvastain calcium

(76) Inventors: Laszlo Czibula, Budapest (HU); Laszlo Dobay, Budapest (HU); Eva Werkne Papp, Budapest (HU); Ida Deutschne Juhasz, Budapest (HU); Sandorne Balint, Budapest (HU)

Correspondence Address: STERNE, KESSLER, GOLDSTEIN & FOX PLLC, 1100 NEW YORK AVENUE, N.W., WASHINGTON, DC, 20005, US

(21) Appl. No.: 10/550,415

(22) PCT Filed: Mar. 23, 2004
(86) PCT No.: PCT/HU04/00026

§ 371 Date: Nov. 13, 2006

(30) Foreign Application Priority Data
Mar. 24, 2003 (HU) P0300761

(51) Int. Cl. C07D 207/337

(52) U.S. Cl. 548/537

(57) ABSTRACT

The invention relates to a new process for the synthesis of amorphous atorvastatin calcium, which consists of dissolving the salt of the formula (I) of atorvastatin acid formed with a basic amino acid (I); in a mixture of water and a water miscible organic solvent, adding an aqueous solution of a water soluble calcium salt to the solution and isolating the so obtained entirely amorphous atorvastatin calcium of high purity by filtration.

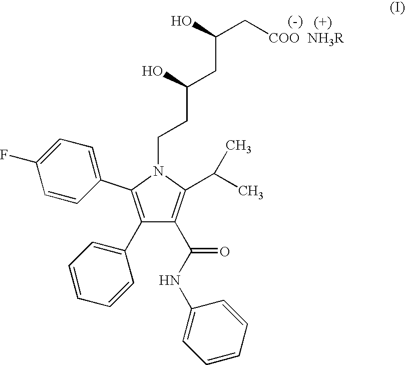

The invention relates to a new process for the synthesis of amorphous atorvastatin calcium.

Atorvastatin calcium (its chemical name is: [R-(R*,R*)]-2-(4-fluorophenyl)-β,δ-dihydroxy-5-(1-methyl-ethyl)-3-phenyl-[(amino)-carbonyl]-1H-pyrrol-1-heptanoic acid hemi-calcium salt) is known as a very efficient cholesterol level decreasing compound acting as an inhibitor of 3-hydroxy-3-methyl-glutamine-coenzim “A” reductase enzyme.

Atorvastatin calcium—as a new chemical entity—is first described in the U.S. Pat. No. 5,273,995. However, there is no information about the crystal form of the product in this description. Later on four different crystal modifications of atorvastatin calcium have been described in the literature, the morphological characterization and the synthesis of which are given in patent applications WO 97/03958 and WO 97/03959.

It is important to know that the amorphous atorvastatin calcium, which became known meanwhile, has better bioavailability than the crystalline forms. Unambiguous data support, that amorphous modification has more favourable features, for example better dissolution properties, than the crystalline one [see: Konno I.: Chem. Pharm. Bull., 38, 2003-2007 (1990)].

According to the above mentioned facts there is a need for elaborating a process for the synthesis of amorphous atorvastatin calcium. The common feature of the known procedures is that the entirely amorphous form of the atorvastatin calcium is obtained from one of the crystalline forms, or a mixture of the different crystalline forms or a mixture of the partially crystalline and partially amorphous form.

According to the patent application WO 97/07960 the amorphous atorvastatin calcium is obtained from the so-called crystal form I, in an organic solvent, which does not contain hydroxy group—for example tetrahydrofuran or a mixture of tetrahydrofuran and toluene—applying complicated, tiresome technology of several days.

According to the patent application WO 00/71116 any form of crystalline atorvastatin calcium is dissolved in a solvent, which does not contain hydroxy group (for example THF), then an apolar solvent is added (for example hexane, cyclohexane or heptane) to give the amorphous product, which is isolated by filtration.

According to an other solution (see: patent application WO 01/28999) the crude atorvastatin calcium—in contrast to the above procedures—is dissolved in a C₂₋₄ alcohol or a mixture of alcohols at the boiling point, the solution is cooled and the amorphous product is filtered.

According to the patent application Number of WO 01/42239 the crystal form I—which is most difficult to obtain—is transformed into amorphous atorvastatin calcium the following way the crystalline form is dissolved in a solvent (so-called type 1), for example methanol, ethanol or acetone, and from this very dilute solution the product is precipitated by addition of an other solvent (so-called type 2), for example ether.

The disadvantages of the above procedures are that the applied starting material, the atorvastatin calcium, is obtained by a complicated, long technology and laborious isolation process, the used dilute solutions require large amount of solvents and the isolation of the amorphous product is very tiresome.

Our aim was to elaborate a simple, economical, industrially applicable process for the synthesis of entirely amorphous atorvastatin calcium of high purity.

In our experiments surprisingly it was found that entirely amorphous atorvastatin calcium of high purity can be obtained from some basic salts of atorvastatin of formula (I): 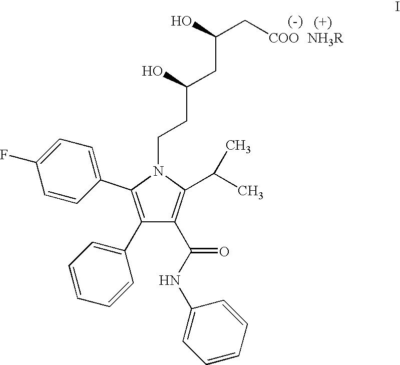
wherein the meaning of R is the compound of formula (II) or (III) 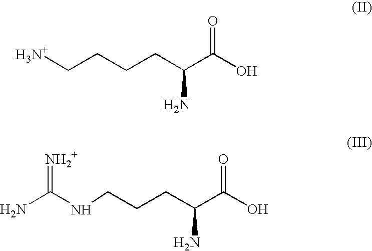
in concentrated, aqueous solution—without isolation or separation of the free acid—in one step, with simple technology. This result was unexpected, because according to the literature data (see for example the patent application WO 97/03959) in the presence of water always one of the crystalline polymorph modifications of the product was obtained. The observation of the present invention results in that the desired product can be synthesized—in contrast to the known procedures—in more concentrated solutions (10-15 w/w %) directly from one of the basic salts of the atorvastatin acid.

According to the above mentioned facts the invention relates to a process for the synthesis of amorphous atorvastatin calcium, which consists of dissolving the salt of the formula (I) of atorvastatin acid formed with a basic amino acid—wherein the meaning of R is the compound of formula (II) or (III)— in a mixture of water and a water miscible organic solvent, adding an aqueous solution of a water soluble calcium salt to the solution and isolating the so obtained entirely amorphous atorvastatin calcium of high purity by filtration.

The salts of atorvastatin acid formed with basic amino acids used in the synthesis according to our invention are atorvastatin L-lysine or atorvastatin L-arginine salts.

The water soluble calcium salts used in the process according to our invention are preferably calcium acetate or calcium chloride.

The water miscible organic solvents used in the process according to our invention are preferably methanol, ethanol, isopropanol or acetone.

According to the process of our invention the desired amorphous final product can be obtained in good yield, chemically pure, with simple technology. The chemical purity of the obtained product was proved by HPLC method, which is described in example 4A and an HPLC chromatogram is also shown. From the chromatography it can be seen that the purity of the product is 99.90 w/w %, it is suitable for producing a pharmaceutical composition. The obtained product contains only three by-products and the amount of each is less than 0.1 w/w %.

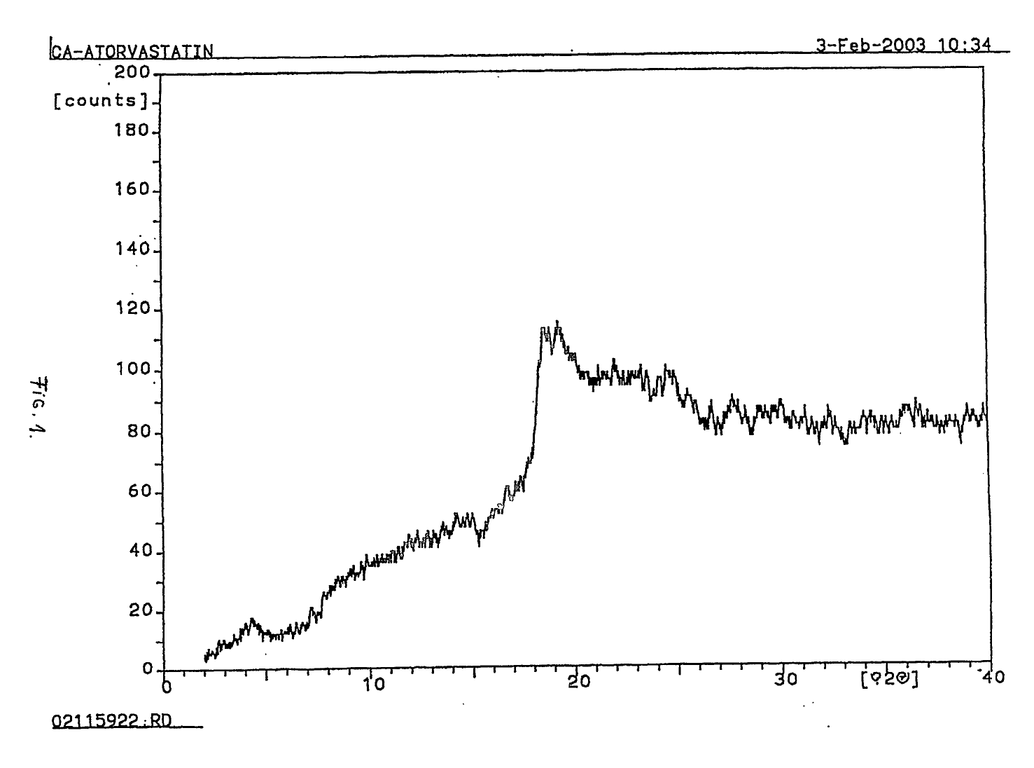

The morphological purity of the product obtained according to the process of our invention was checked by X-ray diffraction method. The so obtained powder diagram (see FIG. 1) proved that the product obtained according to the process of our invention is in 100 w/w % amorphous.

The solvent used in the process of our invention is a mixture of water and a water miscible organic solvent, preferably methanol, ethanol, isopropanol or acetone.

The process of our invention is illustrated in details by the following not limiting examples:

### EXAMPLES

### Example 1

Synthesis of Amorphous Atorvastatin Calcium

16 g of L-lysine salt of atorvastatin is dissolved in a mixture of 437 ml of distilled water and 91.5 ml of ethanol at 35-40° C., the solution is filtered and a filtered solution of 1.92 g of calcium acetate hydrate in 20 ml of distilled water is added. The mixture is cooled to 0-5° C. and filtered immediately, the product is washed with a 5:1 mixture of water and ethanol (2×15 ml), and dried at max. 50° C.

The obtained product is 12.9 g (yield: 98 w/w %)

The chemical purity of the product is: 99.92%
Total amount of impurities is: below 0.08%

The morphological purity of the product (according to X-ray diffraction study):

100% amorphous (see FIG. 1)

### Example 2

Synthesis of Amorphous Atorvastatin Calcium

14 kg of L-lysine salt of atorvastatin is dissolved in a mixture of 60 liter of acetone and 60 liter of ion-exchanged water at 15-20° C. A solution of 1.8 kg of calcium acetate hydrate in 18 liter of ion-exchanged water is added to the solution at the same temperature and the obtained suspension is stirred at 15-20° C. for 1 h. The product is centrifuged, washed with a 1:1 mixture of acetone and water (10 liter) and dried at max. 50° C.

Yield: 10.1 kg (88%) amorphous atorvastatin calcium

The chemical purity of the product is: 99.87% (see Example 5)
Total amount of impurities is: below 0.13%
3 individual impurities: 0.03; 0.04 and 0.06%

The morphological purity of the product (according to X-ray diffraction method): 100% amorphous

### Example 3

Synthesis of Amorphous Atorvastatin Calcium

7.3 g (0.01 mol) of L-arginine salt of atorvastatin acid is dissolved in a mixture of 50 ml of distilled water and 20 ml of 2-propanol at 40° C. A solution of 0.9 g of calcium acetate hydrate in 10 ml of distilled water is added to the solution at this temperature and cooled to 0-5° C. The obtained suspension is stirred at 0-5° C. for 1 h, then the product is filtered, washed with a 5:2 mixture of distilled water and 2-propanol (2×10 ml) and dried at max. 50° C.

The obtained product is: 5.1 g (88%) amorphous atorvastatin calcium

The chemical purity of the product is: 99.9%
Total amount of impurities is: below 0.10%

The morphological purity of the product (according to X-ray diffraction method): 100% amorphous

### Example 4

Methods for measuring the purity of products obtained in Example 1-3:

A/HPLC Method:

Colonna: YMC-Pack Pro C18, 150×4.6 mm ID, 5 μm

Eluent:
A: 100 ml of acetonitrile+895 ml of distilled water+
5 ml of 1 M/dm³ TEAP
B: 900 ml of acetonitrile+95 ml of distilled water+5 ml of 1 M/dm³ TEAP

(TEAP: triethylammonium phosphate buffer, Fluka Chemie, cat. no.: 90362)

| | Time (min) | A % | B % |
|---|---|---|---|
| Gradient: | 0 | 100 | 0 |
| | 5 | 40 | 60 |
| | 10 | 40 | 60 |
| | 15 | 15 | 85 |
| | 18 | 5 | 95 |
| | 26 | 5 | 95 |
| | 26.1 | 100 | 0 |
| | 31 | 100 | 0 |

Detection: for 26 min
Wavelength: 215 nm
Temperature: room temperature
Injected volume: 10 μl
Flow-rate: 1.0 ml/min
Samples were dissolved in a mixture of acetonitrile: distilled water 1:1, concentration 0.8 mg/ml
B/X-Ray Diffraction Method
The study was carried out by Philips PW 1840 powder diffractometer using the following parameters:
CuK_{α} radiation: 30 kV, 30 mA
Goniometer speed: 0.05 °2θ/s
Sensitivity: 2×10³ cps
T.C.: 5 seconds
Gap width: 0.05 mm
   According to the obtained powder diagram (FIG. 1) the sample is amorphous, there are no diffraction peaks in the powder diagram

### Example 5

HPLC chromatogram of the product obtained in Example 2:

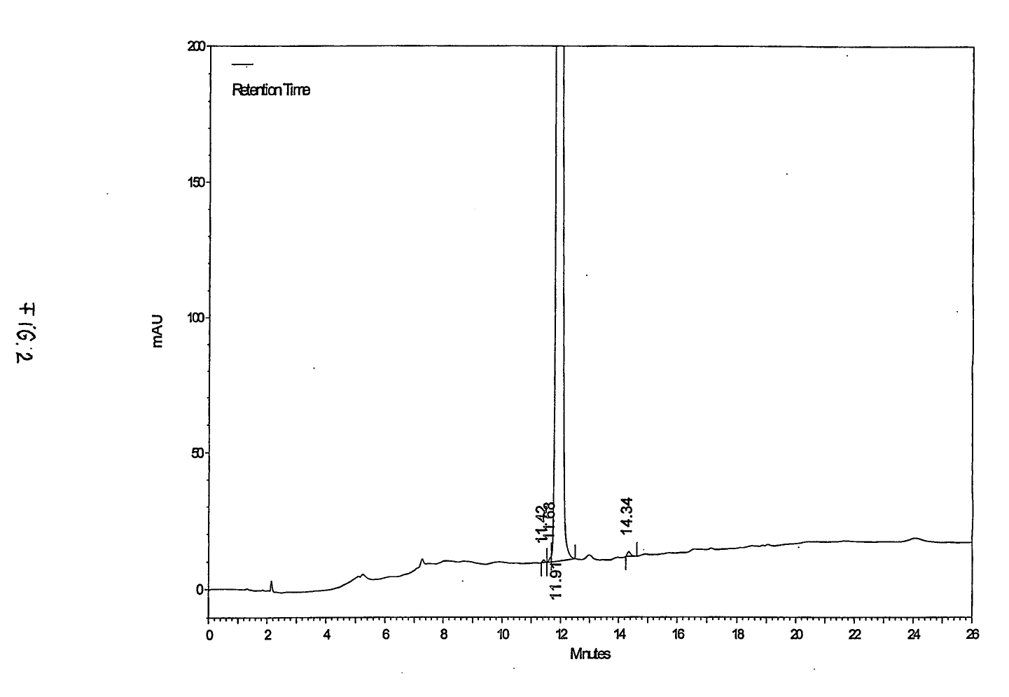

(See: FIG. 2)

Data of Chromatogram:

11.423 min, RRT: 0.96: 0.03 area %
11.683 min, RRT: 0.98: 0.04 area %
14.342 min, RRT: 1.20: 0.06 area %

What we claim is:

1. A process for the synthesis of amorphous atorvastatin calcium characterized by dissolving the salt of the formula (I) of atorvastatin acid formed with a basic amino acid 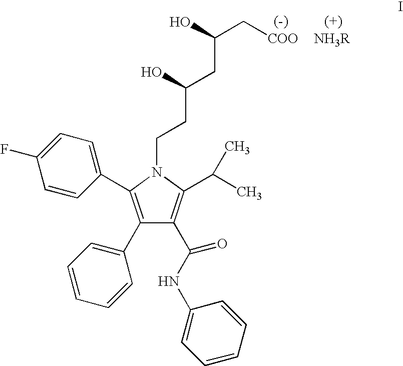
wherein the meaning of R is the compound of formula (II) or (III) 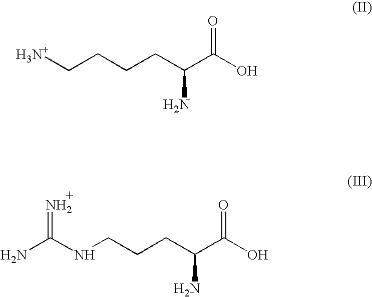
in a mixture of water and a water miscible organic solvent, adding an aqueous solution of a water soluble calcium salt to the solution and isolating the so obtained entirely amorphous atorvastatin calcium of high purity by filtration.

2. The process according to claim 1, characterized by using calciuim acetate or calcium chloride as water soluble calcium salt.

3. The process according to claims 1 and 2, characterized by using methanol, ethanol, isopropanol or acetone as water miscible organic solvent.
